(19) United States
(12) Patent Application Publication
(10) Pub. No.: US 2004/0181154 A1
(43) Pub. Date: Sep. 16, 2004

(54) Ultrasonic diagnostic imaging devices with fuel cell energy source

(76) Inventors: Roy Peterson, Seattle, WA (US); Lauren Pflugrath, Seattle, WA (US); David Roundhill, Woodinville, WA (US)

Correspondence Address: ATL ULTRASOUND, P.O. BOX 3003, 22100 BOTHELL EVERETT HIGHWAY, BOTHELL, WA, 98041-3003, US

(21) Appl. No.: 10/791,509

(22) Filed: Mar. 2, 2004

Related U.S. Application Data
(60) Provisional application No. 60/454,932, filed on Mar. 13, 2003.

(51) Int. Cl. A61B 8/14

(52) U.S. Cl. 600/459

(57) ABSTRACT

Ultrasonic diagnostic imaging devices are powered by fuel cells providing the continuous production of electrical energy by the direct electrochemical conversion of a hydrogen-based fuel into a flow of current. The ultrasound devices described comprise wireless transducer probes, handheld ultrasound systems, and cart-borne or tabletop ultrasound systems. The fuel for the fuel cells is contained in replaceable containers such as cartridges or ampules. When the fuel is exhausted, the fuel cells are immediately returned to a fully operating condition by replacing the expended unit with a full cartridge or ampule.

This application claims the benefit of Provisional U.S. Patent Application serial No. 60/454,932, filed Mar. 13, 2003.

This invention relates to ultrasonic diagnostic imaging, and more particularly, to ultrasonic diagnostic imaging devices and systems which are powered by fuel cell architectures that allow the system to be easily configured for specific applications and to be easily “recharged.”

Ultrasonic diagnostic imaging systems are commonly used to image a wide variety of organs and tissues within the human body. The safe, non-ionizing energy produced by ultrasound has made ultrasound imaging well suited for applications in abdominal, cardiology, pediatrics, and obstetrics, among others. The advanced integration of the technology used to make ultrasound systems such as micro-machined transducers, high density integrated circuits, and solid state display devices has enabled these systems and devices to be smaller than ever before. For example, U.S. Pat. No. 6,440,076 describes an ultrasound system constructed as a portable table-top unit and U.S. Pat. No. 5,722,412 shows ultrasound systems constructed as handheld units. The smaller devices in turn have become more portable, finding applications outside the hospital in emergency rescue units and with military units in the field. These new operating environments often do not have convenient a.c. power sources, and this situation combined with the smaller sizes has led many of these smaller ultrasound systems to become battery powered. Even components of ultrasound systems such as wireless probes are becoming battery powered, such as those described in U.S. Pat. No. 6,142,946.

Battery powered ultrasound devices present the same needs and limitations of other batter powered devices such a laptop computers and cellphones. It is necessary to keep them fully charged to as great a degree as possible, so that the ultrasound devices will always be ready for extended utilization. Recharging between uses becomes necessary and must become part of the routine of use of the devices. Needless to say, it can be inconvenient or hazardous for the patient when battery power becomes expended in the middle of a diagnostic exam, requiring the patient to return when the ultrasound device is recharged or a serious medical condition to go undiagnosed. Accordingly it is desirable to provide battery power which is convenient, safe, provides extended use, and is rechargeable in a matter of seconds, not hours.

In accordance with the principles of the present invention, diagnostic ultrasound devices are described which are powered by fuel cells. Unlike conventional batteries, fuel cells provide advantages for ultrasound devices such as high power densities, high energy densities, and extended run times. Moreover, the fuels cells of the present invention can be “recharged” in a matter of seconds by simple replacement of the fuel container. The fuel cells of the ultrasound devices of the present invention are safe, light-weight, and clean, thus providing portable energy sources well suited to ultrasound devices.

In the drawings:

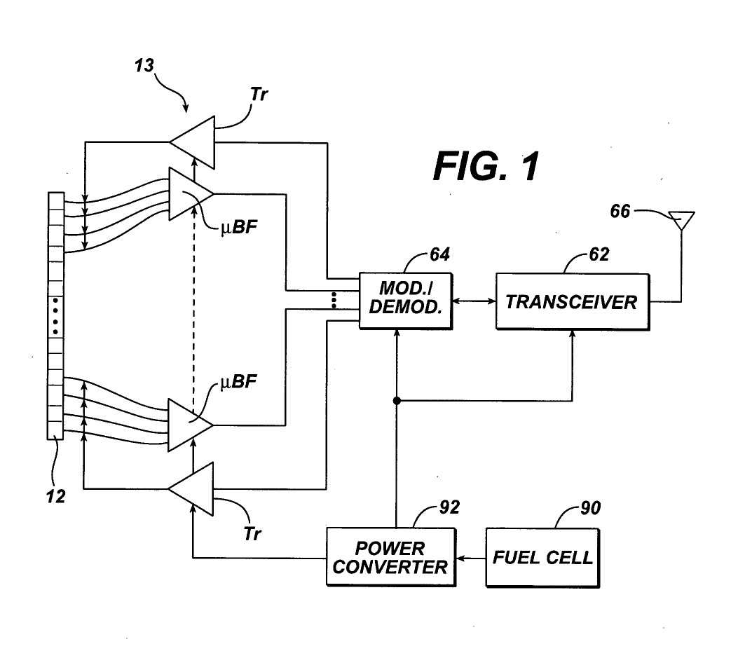

FIG. 1 is a schematic illustration of a wireless ultrasound probe constructed in accordance with the principles of the present invention;

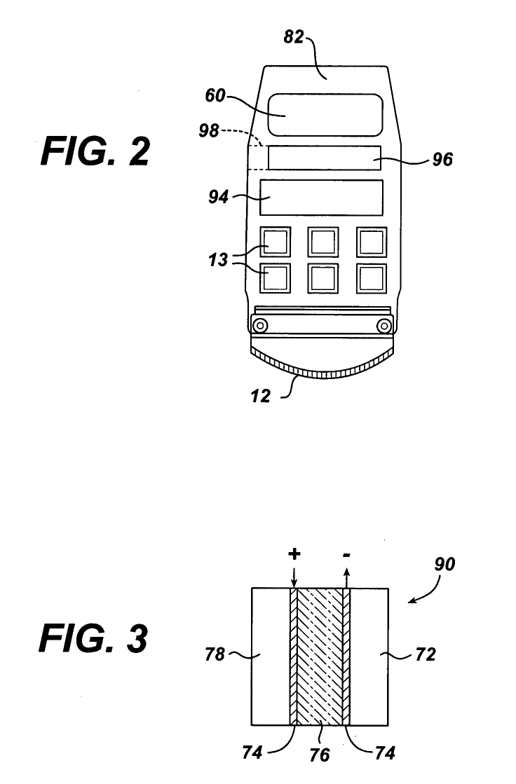

FIG. 2 is a plan view of a wireless ultrasound probe assembly;

FIG. 3 is a schematic illustration of a fuel cell;

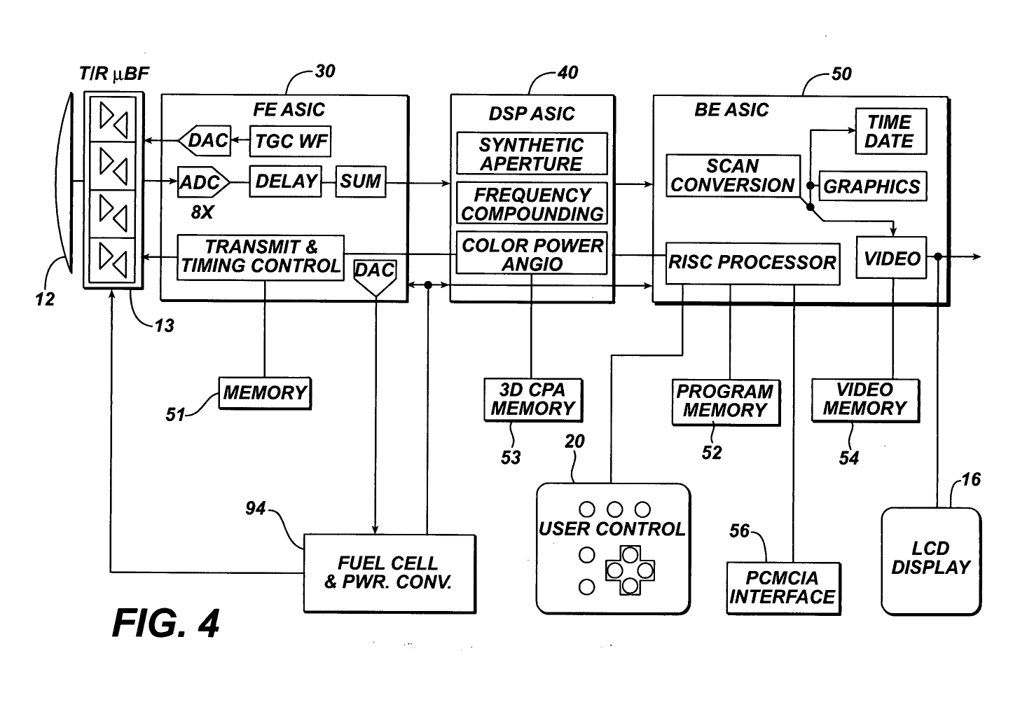

FIG. 4 is a schematic illustration of a handheld ultrasound system constructed in accordance with the principles of the present invention;

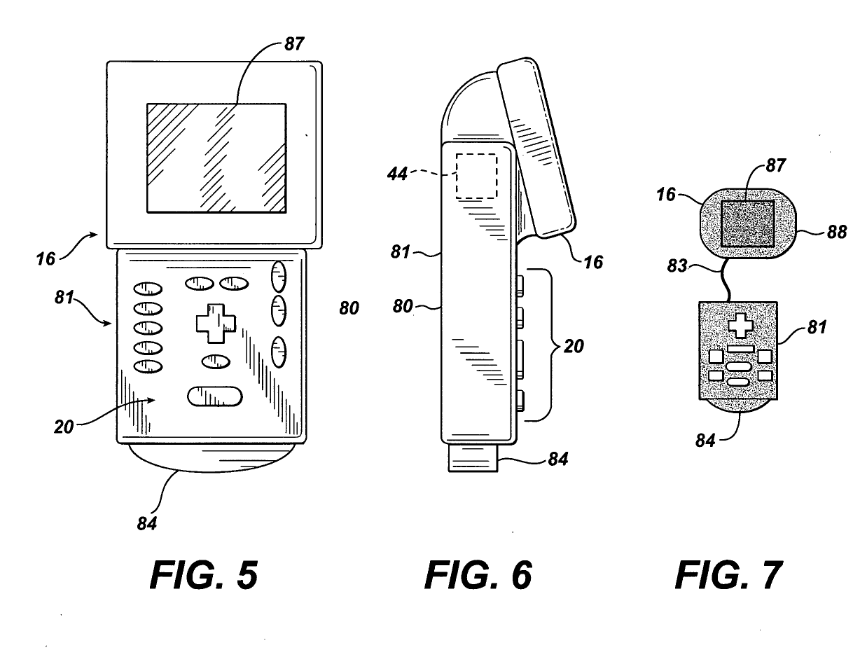

FIGS. 5, 6, and 7 are front and side views of handheld ultrasound systems constructed in accordance with the principles of the present invention;

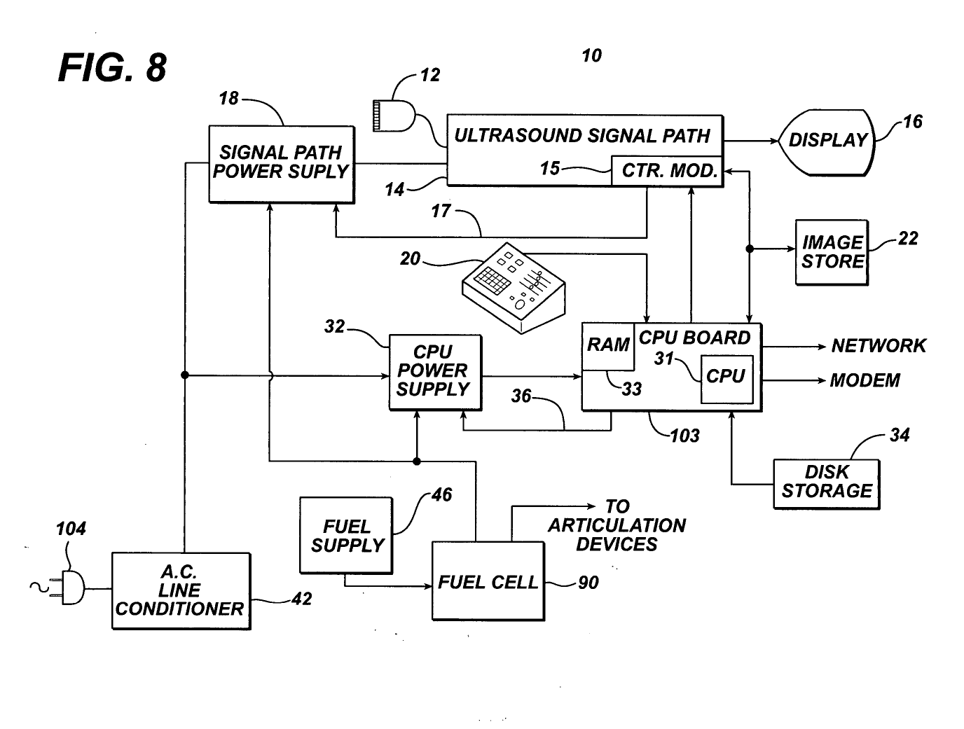

FIG. 8 is a schematic illustration of a cart-borne ultrasound system constructed in accordance with the principles of the present invention; and

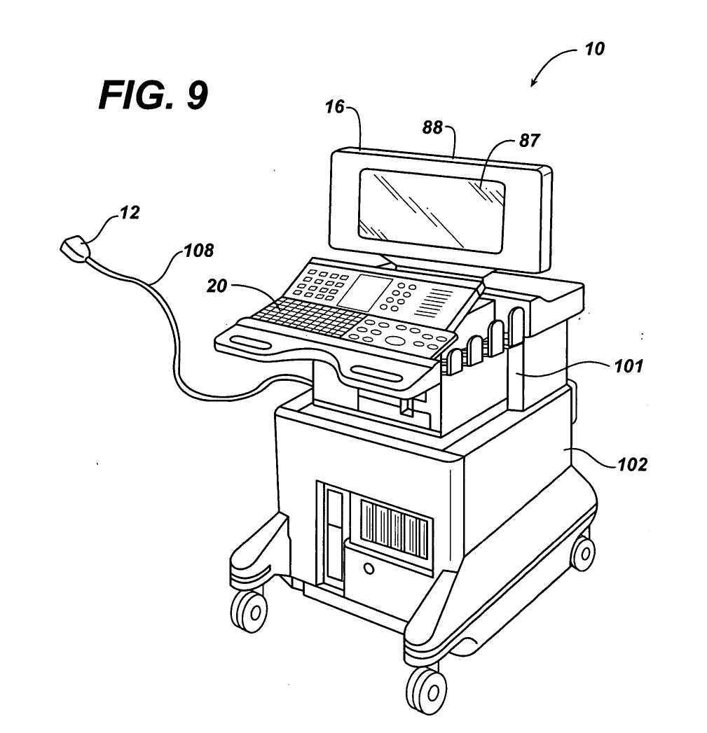

FIG. 9 is a perspective view of a cart-borne ultrasound system.

Referring first to FIG. 1, a wireless ultrasound probe constructed in accordance with the principles of the present invention is shown. The probe includes an array transducer 12 comprising a plurality of transducer elements. The transducer elements are coupled to one or more transmit/receive (T/R) integrated circuits 13. The T/R integrated circuits include a plurality of transmitters Tr. which are coupled to apply actuation signals to selected transducer elements. By selecting the actuation times for the different elements the transducer 12 can transmit steered and focused transmit beams. Echo signals received by the transducer elements are applied to a plurality of microbeamformers (μBF) located on the integrated circuits 13. The microbeamformers, also knows as subarray beamformers, will receive echo signals from a group of transducer elements and perform part of the beamforming process by selectively delaying and combining echo signals. Microbeamformers or subarray beamformers are more fully described in U.S. Pat. Nos. 6,375,617 and 5,997,479.

The partially beamformed signals are prepared for transmission to an ultrasound system processor where the remaining beamforming will be performed and the beamformed signals processed for display. This preparation includes appropriately sequencing and modulating the signals for wireless transmission. This preparation is performed by and modulation/demodulation circuit 64, which may use a multiplexer to sequence the signals for transmission. The partially beamformed signals may also be prepared by time or frequency multiplexing encoding as more fully described in U.S. patent [application Ser. No. 10/091,952, filed Mar. 5, 2002] and entitled “Diagnostic Ultrasonic Imaging System Having Combined Scanhead Connections.” The signals are modulated for example by quadrature modulation, then applied to a transceiver 62 for transmission over an antenna 66 to a receiver and subsequent processing. Details of suitable modulation and transmission schemes are given in U.S. Pat. No. 6,142,946, the contents of which is incorporated herein by reference. The transceiver also 62 receives signals from the base unit. The received signals provide new information as to image formats and transmit and receive timing, focusing changes, and beam characteristic changes. These signals are demodulated by the modulator/demodulator 64 and applied to the T/R integrated circuit 13, where they may be buffered and/or used to change the character of the transmit or receive beams. The probe may also include analog-to-digital and digital-to-analog converters as appropriate for the types of circuitry in the probe and the types of data exchanged with the base unit.

In accordance with the principles of the present invention the wireless probe of FIG. 1. is powered by a fuel cell 90. The fuel cell powered probe is turned on or off by a power-on-off switch (not shown). The fuel cell produces a voltage which is stepped up as necessary by a power converter 92. For instance, piezoelectric transducer elements may require a higher drive voltage than that provided by the fuel cell 90, in which case the power converter 92 will step up the voltage through DC to DC conversion. The power converter may also include a capacitive storage element such as an ultracapacitor to store energy for peak load conditions. Fuel cell power is supplied by the power converter 92 to those elements of the probe requiring electricity, including the integrated circuits 13, the modulator/demodulator 64 and the transceiver 62. Whereas a conventional battery such as a lithium-ion battery produces electricity by an electrochemical process involving acids and metals, a fuel cell produces electricity directly from an ionic separation of hydrogen. In a conventional battery there are two electrodes which are separated by an electrolyte. At least one of the electrodes is generally made of a metal which is converted to another chemical compound during the production of electricity. When this conversion can be reversed, the battery is rechargeable, in which case the recharging current restores ions to the consumed metal. Otherwise, the battery is discharged when the metal is fully consumed and there is no further material for the chemical reaction. In a fuel cell the electrodes are not consumed. Instead, a hydrogen-based fuel is used directly at one electrode where electrons are separated for the flow of current. The hydrogen protons react with an oxidant such as oxygen at the other electrode, thereby producing electricity and the release of water and heat. The fuel cell continues to produce electricity as long as fuel is supplied to it. In some instances the fuel for the fuel cell may need to be reformed into a form in which the hydrogen electrons can be readily separated. Current fuel cells have three to five times the specific energy of comparable lithium-ion batteries and produce six to seven times the energy per unit mass as lithium-ion batteries, with an upper practical limit of approximately thirty. Furthermore, unlike most rechargeable batteries, the fuel cell has no long term memory which degrades the performance of the fuel cell over time.

The fuel cell 90 is formed as schematically illustrated by FIG. 3. The fuel cell has two electrodes, an anode 72 and a cathode 78. The anode and the cathode are separated by an electrolyte 76. Each electrode is coated with a catalyst 74. Alternatively, the electrodes may be formed from porous materials that are laced with the catalyst. There are over half a dozen different types of fuel cells which may be designed for differently sized cells and power output. The electrolytes used in these different types of cells can be solids or liquids and include substances such as phosphoric acid, alkali carbonates, yttria stabilized zirconia and ion exchange membranes. In a currently preferred embodiment of the present invention, an organic ion exchange membrane is used for the electrolyte 76. The catalysts currently used for fuel cells include platinum, platinum-ruthenium alloys, nickel, and perovskites. Platinum is the currently preferred catalyst 74 in the embodiment of FIG. 3. Fuels used for fuel cells preferably are those which are high in hydrogen content, such as gasoline, natural gas, propane or methanol. The current preferred embodiment uses methanol or alcohol for fuel.

In use, the fuel comes into the anode side 72 of the fuel cell, where the catalyst 74 promotes separation of hydrogen molecules of the fuel into protons, electrons, and possibly other byproducts such a carbon dioxide. The negatively charged hydrogen electrons are repelled by the anode and flow to an external circuit as indicated by the “-” arrow at the top of the anode, thereby providing current flow for the probe electrical components. The hydrogen protons are conducted through the polymer exchange membrane 76 to another platinum catalyst 74 at the cathode. Here the protons combine with the electrons from the external circuit at the “+” electrode and oxygen, which may be supplied from the air. The electrons, hydrogen protons, and oxygen combine to produce heat and water and possibly other byproducts such as carbon dioxide gas. The water may be released as water vapor, or reused in a mixture with the fuel at the anode side of the fuel cell. The direct conversion of fuel into electricity enables fuel cells to achieve substantially higher efficiencies in the conversion of hydrocarbon fuels than do more traditional processes such as internal combustion engines. Fuel cells can attain efficiencies of 35% to 90%, depending upon the degree of utilization of the heat produced by the cells.

While the fuel cell 90 is shown in a rectangular illustration in FIG. 3, other physical layouts are possible such as a cylindrical configuration, in which the fuel supply is in the center of the cell and surrounded by the electrodes, catalysts and electrolyte.

FIG. 2 illustrates the packaging of components of the wireless probe of FIG. 1 on a printed circuit board substrate 82. The package of FIG. 2 is contained in a probe case (not shown). Mounted at one end of the printed circuit board 82 is the transducer module 12, which includes the transducer array elements and an acoustic backing material. Located adjacent to the transducer array and connected thereto by printed circuit board conductors are the T/R integrated circuits 13. Behind the integrated circuits 13 are the fuel cell and power converter circuit 94. The fuel cell and power converter circuit 94 are electrically connected to the integrated circuits 13 and the array transducer. Located in proximity to the fuel cell and connected thereto by the appropriate conduit is a fuel ampule compartment 98 containing a fuel ampule 96. In a preferred embodiment the fuel ampule contains methanol or alcohol or a similar compound which is used to fuel the fuel cell. When the fuel in the ampule is exhausted, the ampule is removed and replaced by a full ampule. Thus, “recharging” the fuel cell only requires the brief time necessary to replace the fuel ampule. Located at the rear of the printed circuit board 82 are the modulator/demodulator and transceiver 60 for wireless communication with the external ultrasound processing and display system.

A typical fuel cell such as described for the above embodiment is capable of producing several watts of power. This is more than sufficient for most wireless probes, which can exhibit power requirements of approximately 750 mW to 2 watts. A typical wireless probe may consume 200 mW by the microbeamformers and 500-900 mW by the wireless transceiver system. A microcontroller and a signal processing DSP may consume 100 mW each, and A/D converters may consume 300 mW. With an efficiency factor of 80%, power consumption of the wireless probe can be in the 1-2 Watt range.

FIG. 4 schematically illustrates a second embodiment of the present invention, which is a handheld ultrasound system constructed in accordance with the principles of the present invention. The handheld ultrasound system comprises a transducer array 12. Either a flat or curved linear array can be used, which can be a one dimensional or a 1.5D array for two dimensional imaging, or a two dimensional array for three dimensional imaging. In a preferred embodiment the array is a curved array, which affords a broad sector scanning field. While the preferred embodiment provides sufficient delay capability to both steer and focus a flat array such as a phased array, the geometric curvature of the curved array reduces the delay requirements on the beamformer. The elements of the array are connected to a transmit/receive ASIC 13 which drives the transducer elements and receives echoes received by the elements by means of transmitters and receivers or microbeamformers. The transmit/receive ASIC 13 also controls the transmit and receive apertures of the array 12 and the gain of the received echo signals. The transmit/receive ASIC is preferably located within inches of the transducer elements, preferably in the same enclosure, and just behind the transducer 12.

Echoes received by the transmit/receive ASIC 13 are provided to the adjacent front end ASIC 30, which beamforms the echoes from the individual transducer elements or the partially beamformed signals from microbeamformers into scanline signals. Instead of ASICs, an embodiment of the present invention may alternatively use DSPs or FPGAs for the ASIC circuitry. The front end ASIC 30 also controls the transmit waveform, timing, aperture and focusing. In the illustrated embodiment the front end ASIC 30 provides timing signals for the other ASICs, time gain control, and monitors and controls the power applied to the transducer array, thereby controlling the acoustic energy which is applied to the patient and minimizing power consumption of the unit. A memory device 51 is connected to the front end ASIC 30, which stores data used by the beamformer.

Beamformed scanline signals are coupled from the front end ASIC 30 to the adjacent digital signal processing ASIC 40. The digital signal processing ASIC 40 filters the scanline signals and in the preferred embodiment also provides several advanced features including synthetic aperture formation, frequency compounding, Doppler processing such as power Doppler (color power angio) processing, and speckle reduction.

The ultrasound B mode and/or Doppler information is then coupled to the adjacent back end ASIC 50 for scan conversion and the production of video output signals. A memory device 53 is coupled to the digital signal processing ASIC 40 to provide storage used in three dimensional power Doppler (3D CPA) imaging. The back end ASIC adds alphanumeric information to the display such as the time, date, and patient identification. A graphics processor overlays the ultrasound image with information such as depth and focus markers and cursors. Frames of ultrasonic images are stored in a video memory 54 coupled to the back end ASIC 50, enabling them to be recalled and replayed in a live Cineloop® real-time sequence. Video information is available at a video output in several formats, including NTSC and PAL television formats and RGB drive signals for an LCD display 16 or a video monitor.

The back end ASIC 50 also includes the central processor for the ultrasound system, a RISC (reduced instruction set controller) processor. Alternatively the processor may be an FPGA or a microprocessor. The RISC processor is coupled to the front end and digital signal processing ASICs to control and synchronize the processing and control functions throughout the hand-held unit. A program memory 52 is coupled to the back end ASIC 50 to store program data which is used by the RISC processor to operate and control the unit. The back end ASIC 50 is also coupled to a data port configured as a PCMCIA interface 56. This interface allows other modules and functions to be attached to the hand-held ultrasound unit. The interface 56 can connect to a modem or communications link to transmit and receive ultrasound information from remote locations. The interface can accept other data storage devices to add new functionality to the unit, such as an ultrasound information analysis package.

The RISC processor is also coupled to the user controls 20 of the unit to accept user inputs to direct and control the operations of the hand-held ultrasound system.

Power for the hand-held ultrasound system in a preferred embodiment is provided by a fuel cell and power converter circuit 94. The power converter distributes the required voltages to the ASICS, memory devices and the LCD display, and receives a control signal from a DAC on the front end ASIC 30 which indicates the drive voltage required by the transducer array. The fuel cell and power converter 94 includes a DC converter to convert the low fuel cell voltage to a higher voltage which is applied to the transmit/receive ASIC 20 to drive the elements of the transducer array 10. The fuel cell and power converter 94 includes an alternate power sensor which senses when the ultrasound system is being powered from an external a.c. source and switches the fuel cell source off or on as appropriate. The fuel cell and power converter 94 is coupled to the LCD display 16 to display an indication of the amount of fuel remaining the cell's fuel supply, and to provide an insistent warning when the fuel is about to become exhausted, which may interrupt an ongoing ultrasonic examination. The fuel cell and power converter 94 may also include a capacitor or ultracapacitor to store charge for peak demand conditions. In this embodiment as well as other embodiments described herein the fuel cell and power converter may be a unitary module. Rather than just replace the fuel container, the entire integrated fuel cell and fuel supply may be replaced when replenishing the fuel supply.

FIGS. 5 and 6 illustrate a one piece unit 80 for housing the ultrasound system of FIG. 4. The front of the unit is shown in FIG. 5, including an upper section 16 which includes the LCD display 87. The lower section 81 includes the user controls 20. The user controls enable the user to turn the unit on and off, select operating characteristics such as the mode (B mode or Doppler), color Doppler sector or frame rate, and special functions such as calculation functions or three dimensional display. The user controls also enable entry of time, date, and patient data. A four way control, shown as a cross, operates as a joystick to maneuver cursors on the screen or select functions from a user menu. Alternatively a mouse ball or track pad can be used to provide cursor and other controls in multiple directions. Several buttons and switches of the controls are dedicated for specific functions such as freezing an image and storing and replaying an image sequence from the Cineloop memory.

At the bottom of the unit 80 is the aperture 84 of the curved transducer array 12. In use, the transducer aperture is held against the patient to scan the patient and the ultrasound image is displayed on the LCD display 87.

FIG. 6 is a side view of the unit 80, showing the depth of the unit. On the side of the unit is an opening 44 in the housing to the compartment 98 in which the fuel cartridge or ampule 96 of fuel for the fuel cell is located. The unit 80 is approximately 20.3 cm high, 11.4 cm wide, and 4.5 cm deep. This unit contains all of the elements of a fully operational ultrasound system with a curved array transducer probe, in a single package weighing less than five pounds. The transducer array, ASICs, and associated power supply and control circuitry can consume approximately 7.5 watts, and the display can consume an additional 5.3 watts, for at total of less than thirteen watts. When scanning is suspended during image “freeze,” power consumption can be reduced to approximately 6.5 watts. When the unit is in a suspend or “sleep” mode, power consumption can drop to less than one watt.

FIG. 7 illustrates a second packaging configuration in which the ultrasound system is housed in two separate sections. A lower section 81 includes the transducer array, the electronics of the signal path through to a video signal output, and the user controls. This lower section is shown in FIG. 7 with the curved transducer array aperture 84 visible at the bottom. The lower section measures about 11.4 cm high by 9.8 cm wide by 2.5 cm deep. This unit 81 has approximately the same weight as a conventional ultrasound probe. This lower section is connected to an upper section 88 as shown in FIG. 7 by a cable 83. The upper section 88 includes an LCD display 87 and the fuel cell, fuel supply and power converter circuitry. The cable 83 couples video signals from the lower unit 81 to the upper unit for display, and provides power for the lower unit from the fuel cell and power converter 94. This two part unit is advantageous because the user can maneuver the lower unit and the transducer 84 over the patient in the manner of a conventional scanhead, while holding the upper unit in a convenient stationary position for viewing. By locating the fuel cell and circuitry 94 in the upper unit, the lower unit is lightened and easily maneuverable over the body of the patient.

Other system packaging configurations will be readily apparent. For instance, the front end ASIC 30, the digital signal processing ASIC 40, and the back end ASIC 50 could be located in a common enclosure, with the beamformer of the front end ASIC connectable to different connectable array transducers. This would enable different transducers to be used with the digital beamformer, digital filter, and image processor for different diagnostic imaging procedures. A display could be located in the same enclosure as the three ASICS, or the output of the back end ASIC could be connected to a separate display device. Further details of handheld ultrasound systems may be found in U.S. Pat. No. 5,722,412.

FIG. 8 is a schematic illustration of a cart-borne ultrasound system constructed in accordance with the principles of the present invention. The components of a typical ultrasound system are shown at the top of the drawing, including a scanhead or transducer 12, an image display 16, and the ultrasound signal path 14 which connects the transducer and the display. The ultrasound signal path will typically include a beamformer which controls the transmission of ultrasonic waves by the transducer 12 and forms received echo signals into steered and focused beams, a signal processor which processes coherent echo signals in the desired mode of display, e.g., B mode, Doppler mode, harmonic or fundamental mode, and an image processor which produces image signals of the desired format from the processed echo signals, such as for a 2D or 3D image or spectral Doppler display. The ultrasound signal path is controlled in a coordinated manner by a system controller which responds to user commands and dictates the overall scheme of functionality of the ultrasound signal path. For instance, the system operator may enter a command on the user control panel 20 to request two dimensional colorflow imaging using a certain scanhead. The system controller would respond to this command by conditioning the beamformer to operate and control the desired scanhead, initializing the signal processor to Doppler process the received echo signals, and setting up the image processor to produce a grayscale B mode image with flow shown as a color overlay.

The source of energy for a cart-borne or tabletop ultrasound system is generally a.c. line voltage accessed by a plug 104. The a.c. power is filtered and rectified by an a.c. line conditioner 42, which produces a DC supply voltage such as 48 volts. This voltage is supplied to a signal path power supply 18, which supplies power to the scanhead 12 and ultrasound signal path 14. The a.c. line conditioner provides two other functions, which are to sense and respond to different a.c. power sources and to provide power factor correction which matches current and voltage phases to prevent instantaneous current spikes during cycles of the a.c. power. The a.c. line conditioner will sense whether the plug 104 is connected to 110 volt, 60 Hz power or 220 volt 50 Hz power, for instance, and will respond to configure the line conditioner to produce the required 48 VDC from either a.c. source. Power factor correction will cause the ultrasound system to use power more efficiently by appearing as a more resistive rather than reactive load to the a.c. power system. The power supply 18 is a DC to DC converter, which supplies a number of DC voltages for different components and modules of the ultrasound system. For instance, a high voltage is supplied as a drive voltage for the ultrasonic transducer, and lower level voltages are supplied to the digital processing circuitry of the system. The signal path power supply 18 is generally capable of providing 1000 watts or more of power to a cart-borne ultrasound system.

In the embodiment of FIG. 8 a CPU board 103 is coupled to the ultrasound signal path 14 which controls the powering up and powering down of the ultrasound signal path. The functions of the CPU board discussed below may, in a particular embodiment, be integrated into the system controller of the ultrasound signal path and be performed there. In FIG. 8 a separate CPU board is shown for ease of illustration and understanding. The CPU board 103 may comprise an off-the-shelf motherboard such as an ATX form factor motherboard with a system core chipset and basic input/output (BIOS) software. BIOS is code that runs from a non-volatile memory such as a PROM or flash storage device and stays resident on the CPU board. The BIOS software boots the CPU from a cold power-up and launches the operating system. The BIOS software performs such functions as checking basic hardware operability and hardware resources available. Vendors of BIOS software include Phoenix, Award, and American Megatrends. The CPU board includes a CPU processor 31 (sometimes referred to herein as the CPU) which may be a microprocessor such as the microprocessors available from Intel, Advanced Micro Devices, or Motorola, or a processor of more limited capability such as a reduced instruction set (RISC) processor as discussed in the previous embodiment. The CPU board includes a random access memory (RAM) 33 which enables the CPU to run an operating system software program (OS) resident on nonvolatile disk storage 34. The OS is operated to control various operating aspects of the ultrasound signal path 14, display 16 and peripheral devices connected to the ultrasound system such as printers and recorders, as described below. The OS refers to the platform software that tends to housekeeping functions and provides an interface to launch application software. Operating system software includes DOS, Windows95-2000, Windows CE and NT, Solaris, and OS2. Any software that is not an OS and performs a given task is referred to as application software. Examples of application software includes word processor software, spreadsheet software, communication or analysis software, and the custom software that runs an ultrasound machine. In the illustrated embodiment the CPU board is coupled to the ultrasound signal path 14 by way of a control interface shown as control module 15 of the ultrasound signal path 14. When the functionality of the CPU board is integrated into the ultrasound signal path, the need for this interface may be partially or wholly eliminated.

The CPU board may be powered by the signal path power supply 18, however, in the illustrated embodiment the CPU board 30 is powered by its own CPU power supply 32. The CPU power supply has a lower capacity than that of the power supply 18, and may for instance be a 250 watt power supply. The CPU power supply 32, like the power supply 18, is a DC to DC converter which converts the voltage level supplied by the a.c. line conditioner to the DC voltages required by the CPU board 30 and, preferably, also the disk storage 34. The CPU power supply is coupled to the a.c. line conditioner and is energized in the same manner as the power supply 18.

In accordance with the principles of the present invention, the ultrasound system includes a fuel cell 90 which provides a backup source (or, in some instances, a primary source) of power to the signal path power supply 18 and the CPU power supply 32. The fuel cell 90 is fueled by a fuel supply 46 coupled to the fuel cell. The fuel cell 90 is also coupled to the drive motors of articulation devices, when present, by which movable parts of the ultrasound system such as the display 16 and control panel 20 can be raised, lowered, and tilted for the convenience of the operator. This enables the articulated components of the ultrasound system to be moved and adjusted even when the system is not plugged into a wall outlet. Since the power requirements of the cart-borne or tabletop ultrasound system are more substantial than those of the wireless probe or handheld ultrasound system, a fuel cell with a solid or liquid electrolyte and providing greater power than a polymer exchange membrane fuel cell may be used. If motor-driven devices such as motorized articulation devices are connected to the fuel cell, an ultracapacitor will generally be employed to respond to the startup currents of the motors.

An efficiently designed cart-borne ultrasound system can operate at approximately 380 watts, including a CRT display. Peripheral devices may consume another 20-30 watts of power. Use of an LCD or other flat panel display will reduce this power consumption further. If the ultrasound system is configured as a tabletop unit in a form similar to, for instance, that of a laptop computer, the entire unit can exhibit power consumption of approximately 30 watts with a flat panel display.

The ultrasound system of FIG. 8 has connections for a network and/or modem by which diagnostic information obtained by use of the ultrasound system can be remotely stored or shared with others. The network and modem connections also enable information from remote sources to be provided to the ultrasound system, such as electronic mail and reference image libraries as described in U.S. Pat. Nos. 5,897,498 and 5,938,607. In the embodiment shown in FIG. 8 these connections are made from the CPU board 103, although in a particular embodiment they may also be made from the ultrasound signal path 14.

When a conventional cart-borne or tabletop ultrasound system is turned on, it must initialize all of its functionality from a cold start, which can take many minutes to accomplish. Likewise, when the system is turned off, the ultrasound system goes through a lengthy process to power down its various modules and subsystems in an orderly but time consuming sequence. In an embodiment of the present invention, the CPU board 103 is rarely, if ever, completely powered down. The CPU board in a preferred embodiment controls the other components and subsystems of the ultrasound system to be in various suspended states or entirely powered down, and may even itself go into a suspend or low power state, but is selectively available to be restored and to restore the rest of the ultrasound system to full operation in a short or almost instantaneous period of time. Thus, when the system plug is not connected to an a.c. line, as when the cart-borne system is wheeled to another location, it will be desirable to provide at least subsistence-level power to the CPU board 103, which at that time is provided by the fuel cell 90 energizing the CPU power supply 32. When the portable ultrasound system has reached its new destination it can continue to operate while powered by the fuel cell or, it can be plugged into the a.c. line source again. Thus, full system operation may resume immediately without the need to go through an extensive cold-start boot-up procedure.

FIG. 9 illustrates a cart-borne ultrasound system in a perspective view. The diagnostic ultrasound imaging system 10 includes an ultrasound transducer 124 that is adapted to be placed in contact with a portion of a body that is to be imaged. The transducer 12 is coupled to a system chassis 101 by a cable 108. The system chassis 101, which is mounted on a cart 102, includes a keyboard and control panel 20 by which data may be entered into a processor that is included in the system chassis 101. A display monitor 16 having a wide aspect ratio viewing screen 87 in a flat panel housing 88 is placed on an upper surface of the system chassis 101. The a.c. line conditioner 42, the signal path power supply 18, the fuel cell 90 and its fuel supply 46 are located in the lower portion of the cart 102 below the system chassis 101 so as to provide a low center of gravity for the ultrasound system. The fuel supply 46 is accessible for replenishment or replacement from a hinged panel at the rear of the cart (not shown in the view of FIG. 9). A suitable fuel source for a cart-borne system may be compressed hydrogen, for instance. The line cord and plug 104 also extend from the rear of the cart.

What is claimed is:

1. An ultrasonic diagnostic imaging system probe comprising:
an ultrasonic transducer array (12);
an integrated circuit (13) coupled to the ultrasonic transducer array (12) which acts to process or control transducer array signals;
a fuel cell (90) coupled to the integrated circuit (13) for energizing the integrated circuit (13); and
a source of fuel coupled to the fuel cell (90).

2. The ultrasonic diagnostic imaging system probe of claim 1, further comprising a transceiver (62), coupled to the integrated circuit (13), which acts to communicate between the probe (12) and an ultrasound system.

3. The ultrasonic diagnostic imaging system probe (12) of claim 1, wherein the integrated circuit (13) further comprises a beamformer integrated circuit.

4. The ultrasonic diagnostic imaging system probe of claim 1, further comprising a power converter (92), coupled to the fuel cell (90) and the transducer array (12), which produces a stepped up voltage level in response to the power level produced by the fuel cell (90),
wherein the fuel cell (90) further acts to energize the transducer array (12).

5. The ultrasonic diagnostic imaging system probe of claim 1, further comprising a capacitor, coupled to the output of the fuel cell (90), which acts to store energy for peak load conditions.

6. The ultrasonic diagnostic imaging system probe of claim 1, wherein the source of fuel comprises a replaceable fuel cartridge or ampule (96).

7. The ultrasonic diagnostic imaging system probe of claim 6, wherein the fuel cartridge or ampule (96) contains a methanol- or alcohol-based fuel.

8. The ultrasonic diagnostic imaging system probe of claim 1, wherein the fuel cell (90) further comprises an anode (72), a cathode (78), and an ion exchange membrane (76) located between the anode (72) and the cathode (78).

9. The ultrasonic diagnostic imaging system probe of claim 8, wherein the fuel cell (13) further comprises a catalyst metal (74) which acts to promote the separation of hydrogen ions in the fuel cell (90).

10. A handheld ultrasonic diagnostic imaging system comprising:
an ultrasonic transducer array (12);
an integrated circuit (13) coupled to the ultrasonic transducer array (12) which acts to beamform signals produced by or for the transducer array (12), and to process beamformed signals for display;
a display panel (16) coupled to the integrated circuit (13);
a fuel cell (90) coupled to the integrated circuit (13) and the display panel (16) for energizing the integrated circuit (13) and the display panel (16); and
a source of fuel coupled to the fuel cell (90).

11. The handheld ultrasonic diagnostic imaging system of claim 10, further comprising a control panel (20) for operating the handheld ultrasonic diagnostic imaging system; and
a case (81) which houses the integrated circuit (13) and the control panel (20).

12. The handheld ultrasonic diagnostic imaging system of claim 11, wherein the case (80) further houses the display panel (87), the fuel cell (90), and the source of fuel.

13. The handheld ultrasonic diagnostic imaging system of claim 10, further comprising a power converter (92), coupled to the fuel cell (90) and the transducer array (12), which produces a stepped up power level in response to the power level produced by the fuel cell (90),
wherein the fuel cell (90) further acts to energize the transducer array (12).

14. The handheld ultrasonic diagnostic imaging system of claim 10, further comprising a capacitor, coupled to the output of the fuel cell (90), which acts to store energy for peak load conditions.

15. The handheld ultrasonic diagnostic imaging system of claim 10, wherein the source of fuel comprises a replaceable fuel cartridge or ampule (96).

16. The handheld ultrasonic diagnostic imaging system of claim 15, wherein the fuel cartridge or ampule (96) contains a methanol- or alcohol-based fuel.

17. The handheld ultrasonic diagnostic imaging system of claim 10, wherein the fuel cell (90) further comprises an anode (72), a cathode (78), and an ion exchange membrane (76) located between the anode (72) and the cathode (78).

18. The handheld ultrasonic diagnostic imaging system of claim 17, wherein the fuel cell further comprises a catalyst metal (74) which acts to promote the separation of hydrogen ions in the fuel cell (90).

19. The handheld ultrasonic diagnostic imaging system of claim 10, wherein the display panel (87) is further responsive to the source of fuel for the display of the amount of fuel remaining in the fuel source.

20. An ultrasonic diagnostic imaging system comprising:
an ultrasonic transducer array probe (12);
an ultrasound signal path (14) coupled to the transducer array probe (12);
an image display (87) coupled to the ultrasound signal path (14);
a control panel (20) coupled to the ultrasound signal path (14);
a source of a.c. power coupled to energize the ultrasound signal path (14);
a fuel cell (90) coupled to energize the ultrasound signal path (14); and
a source of fuel coupled to the fuel cell (90).

21. The ultrasonic diagnostic imaging system of claim 19, wherein the ultrasound signal path is located in the system chassis (101) of a tabletop ultrasound system.

22. The ultrasonic diagnostic imaging system of claim 19, wherein the ultrasound signal path (14), the image display (16), the control panel (20), the fuel cell (90) and the source of fuel are mounted on a wheeled cart.

23. The ultrasonic diagnostic imaging system of claim 19, wherein the image display (16) is further responsive to the source of fuel for the display of the amount of fuel remaining in the fuel source.

24. An ultrasonic diagnostic imaging system comprising:
an ultrasonic transducer array probe (12);
an ultrasound signal processor coupled to receive signals from the array probe (12);
an ultrasound image processor coupled to receive signals from the signal processor;
an image display (16) coupled to the image processor which acts to display images produced by the image processor; and
a fuel cell (90) and fuel supply unit (46), coupled to provide energy to one or more of the array probe (12), the signal processor, the image processor, and the image display (16),
wherein the fuel cell (90) and fuel supply unit (46) is removable from the diagnostic imaging system for replacement by another fuel cell (90) and fuel supply unit (46) by a user of the ultrasonic diagnostic imaging system.
